(19) United States
(12) Patent Application Publication
(10) Pub. No.: US 2013/0172719 A1
(43) Pub. Date: Jul. 4, 2013

(54) BIOMETRIC INFORMATION DETECTING APPARATUS

(71) Applicant: SEIKO INSTRUMENTS INC., Chiba (JP)

(72) Inventors: Satoshi Sakai, Chiba (JP); Akira Takakura, Chiba (JP)

(73) Assignee: SEIKO INSTRUMENTS INC., Chiba (JP)

(21) Appl. No.: 13/719,315

(22) Filed: Dec. 19, 2012

(30) Foreign Application Priority Data
Dec. 20, 2011 (JP) 2011-278850

(51) Int. Cl. A61B 5/0408; A61B 5/00; A61B 5/04; A61B 5/0245

(52) U.S. Cl. CPC A61B 5/04085; A61B 5/0245; A61B 5/6823; A61B 5/0006; A61B 5/6831; A61B 5/742; A61B 5/04012
USPC 600/382

(57) ABSTRACT

A biometric information detecting apparatus including: a pair of electrodes coming into contact with a surface of a living body; and a circuit board to which a pair of the electrodes are connected and configured to detect biometric information on the basis of a potential difference generated between a pair of the electrodes, the circuit board including an electrode connecting pattern Co which a pair of the electrodes are electrically connected, and a detection circuit unit configured to detect the biometric information on the basis of the potential difference; and a GND pattern provided on the circuit board between the electrode connecting pattern and the detection circuit unit.

### BACKGROUND OF THE INVENTION

1. Technical Field

The present invention relates to a biometric information detecting apparatus configured to detect biometric signals by electrodes attached on the surface of a living body.

2. Description of the Related Art

Examples of a biometric information detecting apparatus of this type include, for example, an apparatus configured to detect an electrocardiac signal generated in association with heart beats and measure a heart rate from the surface of a living body. The biometric information detecting apparatus configured in this manner includes a pair of electrodes configured to detect the electrocardiac signal generating in association with heart beats by being brought into contact with a breast region of a body (the surface of the living body), and a detection circuit board electrically connected to the electrodes and configured to derive the heart rate on the basis of she electrocardiac signal detected by a pair of the electrodes. The detection circuit board includes a detection circuit unit on which electronic components are mounted, and an electrode connecting portion to which the electrodes are electrically connected.

The electrodes and the detection circuit board configured in this manner are integrated with each other and the unified structure is attached on the body using a fixing belt so as to wind around the breast region of the body.

In this manner, the biometric information detecting apparatus is used in a state of being attached on the body, and hence the apparatus is always exposed to moisture such as sweat at the time of measurement. If the moisture cones into contact with the detection circuit board, malfunction may be resulted. Therefore, various waterproofing technologies are proposed.

For example, there is a biometric information detecting apparatus (chest unit) composed of a chest unit holder formed integrally with electrodes and a circuit capsule having the detection circuit board (electronic circuit) and a cell integrated therein, and configured in such a manner that the electrode and the detection circuit board are brought into conduction when the circuit capsule is mounted on the chest unit holder (for example, JP-A-2002-143110).

In this configuration, since the detection circuit board and the cell are built in the circuit capsule, entry of moisture to the detection circuit board is prevented by enhancing the waterproof property of the circuit capsule.

Also, for example, there is a biometric information retooling apparatus (heart beat measuring transmitter) including an operable and closable cell lid configured to seal a cell housing and an opening of the cell housing using a packing in a liquid-tight manner and an openable and closable cell lid cover configured to cover the cell lid on the front side of a case thereof, and a waterproofing resilient member provided between an inner surface of the cell lid cover and the case so as to surround the outside of the outer periphery of the cell lid (for example, see JP-A-2007-143622).

In this configuration, deterioration of the packing on the cell lid due to sweat or the like is prevented by placing the cell lid cover on top of the cell lid, and waterproof property of the biometric information detecting apparatus may be maintained for a long period.

However, in the related art described above, although a superior waterproofing effect is achieved when used under normal conditions, if the biometric information detecting apparatus is exposed to water, moisture enters the case, and the moisture may cause short circuit between the detection circuit unit and the electrode connecting portion of the detection circuit board. In this manner, the related art has a problem that if a potential difference arises between a pair of the electrodes in the state of short circuit between the detection circuit unit and the electrode connecting portion and in this state, an electric current may flow between a pair of the electrodes, which may result in malfunction.

### SUMMARY OF THE INVENTION

It is an aspect of the present application to provide a biometric information detecting apparatus which is capable of preventing malfunction caused by an electric current flowing between a pair of electrodes even when moisture enters a detection circuit board.

There is provided a biometric information detecting apparatus of the application including: a pair of electrodes coming into contact with the surface of a living body; and a circuit board to which a pair of the electrodes are connected and configured to detect biometric information on the basis of a potential difference generated between a pair of the electrodes, the circuit board including an electrode connecting pattern to which a pair of the electrodes are electrically connected, and a detection circuit unit configured to detect biometric information on the basis of the potential difference; and a low potential pattern set to have a potential lower than that of the electrode connecting pattern provided on the circuit board between the electrode connecting pattern and the detection circuit unit.

In this configuration, even when moisture adheres so as to extend across the detection circuit unit and the electrode connecting pattern, the moisture comes to contact with the low potential pattern. Therefore, the potential difference between a pair of the electrodes can be inhibited in comparison with the case where the low potential pattern is not provided, so that an electric current can hardly flow between a pair of the electrodes and hence occurrence of malfunction may be prevented.

The biometric information detecting apparatus of the application is characterized in that the low potential pattern is set to a GND pattern.

In this configuration, the potential of the low potential pattern may be set easily to be lower than the potential of the electrode connecting pattern. Also, the potential difference between a pair of the electrodes may be set to be substantially uniform, so that the flow of the electric current between a pair of the electrodes can be prevented further reliably.

The biometric information detecting apparatus of the application is characterized in that the low potential pattern is provided in the periphery of the electrode connecting pattern.

in this configuration, even when moisture adheres so as to extend across the detection circuit unit and the electrode connecting pattern, the moisture comes to contact with the low potential pattern reliably. Accordingly, the malfunction that may occur on the biometric information detecting apparatus due to moisture may be prevented reliably.

The biometric information detecting apparatus according to the application is characterized in that the circuit board is formed by providing a resist on a pattern layer formed on a substrate body, and a portion on the circuit board corresponding to the low potential pattern is set to be a low potential pattern forming range, and the pattern layer exposed on the circuit board by removing the resist in the low potential pattern forming range or by forming the resist in the low potential pattern forming range to be thinner than other portions is defined as the low potential pattern.

In this configuration, the low potential pattern may be formed easily, and the manufacturing cost of the biometric information detecting apparatus may be reduced.

According to the application, for example, even when moisture adheres so as to extend across the detection circuit unit and the electrode connecting pattern, the moisture comes to contact with the low potential pattern. Therefore, even when the potential difference is generated between a pair of the electrodes, since the potential of the electrode connecting pattern is lowered, the electric current can hardly flow between a pair of the electrodes, so that occurrence of the malfunction may be prevented.

### BRIEF DESCRIPTION OF THE DRAWINGS

FIG. 1 is an explanatory drawing illustrating a state in which a heart rate measuring apparatus according to an embodiment of the invention is attached on a user U;

FIG. 2 is a front view of a heart rate meter body according to the embodiment of the invention;

FIG. 3 is a side view of the heart rate meter body according to the embodiment of the invention;

FIG. 4 is a back view of the heart rate meter body according to the embodiment of the invention;

FIG. 5 is a cross-sectional view taken along the line A-A in FIG. 2;

FIG. 6 is a partial plan view of a circuit board according to the embodiment of the invention; and

FIG. 7 is a block diagram of the heart rate meter body according to the embodiment of the invention.

### DESCRIPTION OF THE PREFERRED EMBODIMENTS

### Biometric Information Detecting Apparatus

Subsequently, an embodiment of the invention will be described with reference to the drawings.

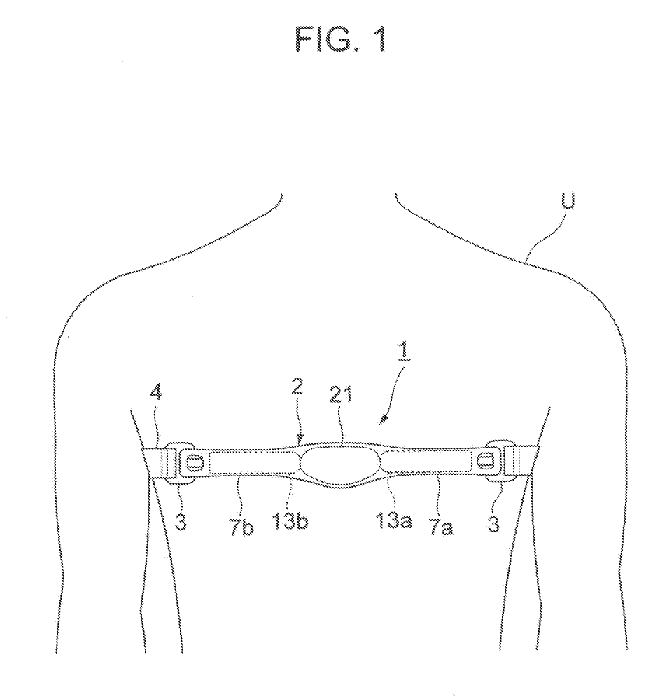

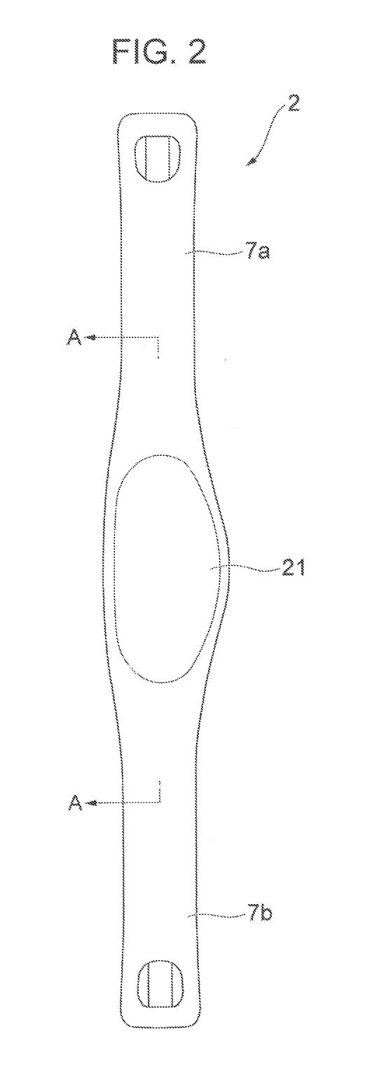

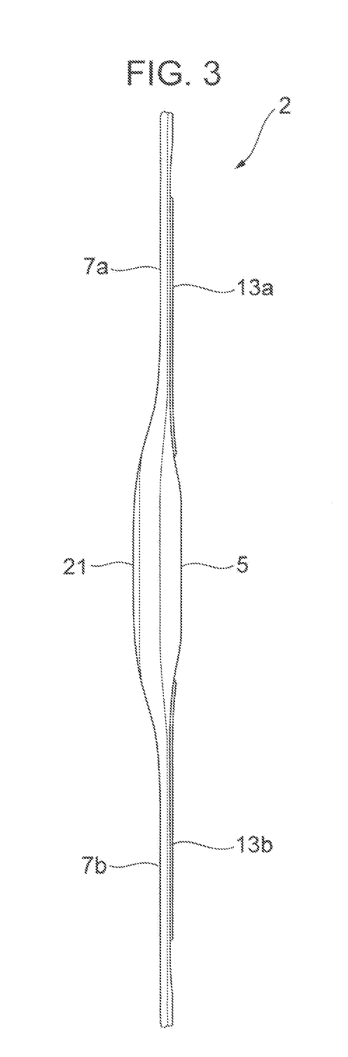

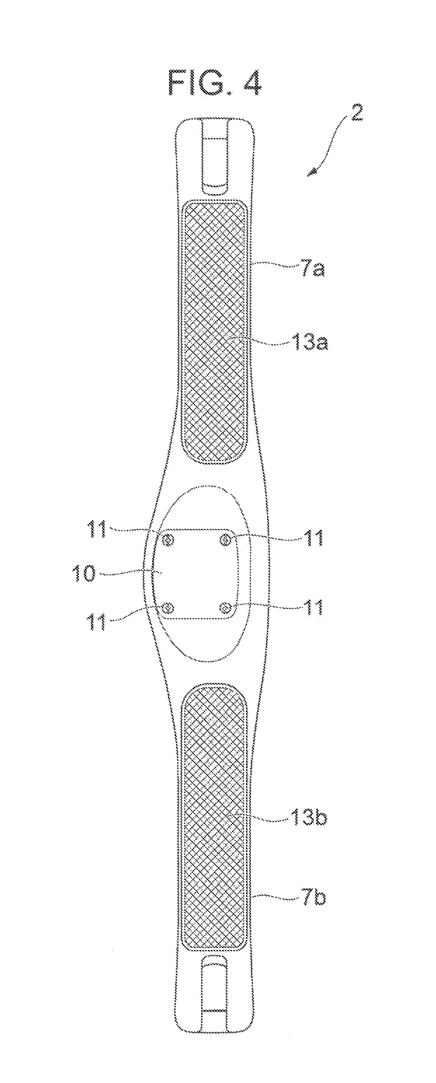

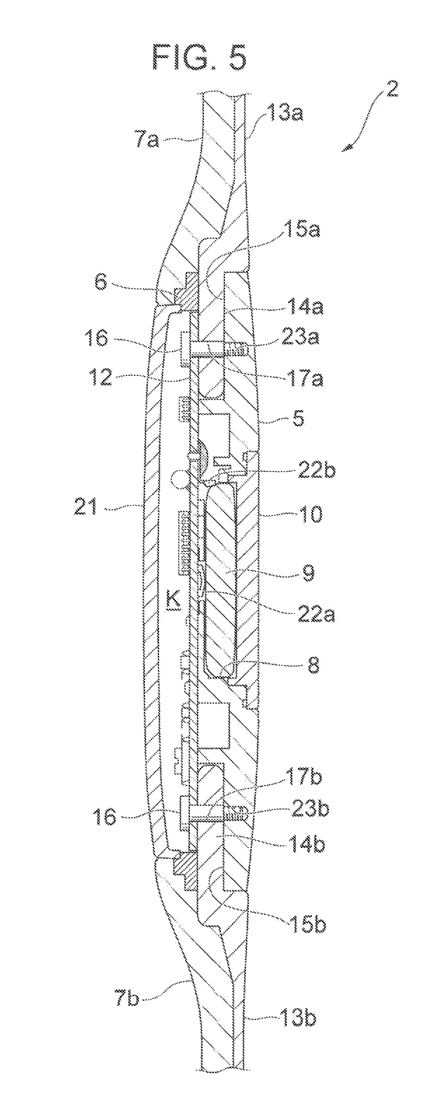

FIG. 1 is an explanatory drawing illustrating a state in which a heart rate measuring apparatus 1 which corresponds to a biometric information detecting apparatus according to the invention is attached on a user U. FIG. 2 is a front view of a heart rate meter body 2 which constitutes the heart rate measuring apparatus 1. FIG. 3 is a side view of the heart rate meter body 2. FIG. 4 is a back view of the heart rate meter body 2. FIG. 5 is a cross-sect local view taken along the line A-A in FIG. 2.

In the description given below, the side of the heart rate measuring apparatus 1 which comes into contact with the user U in a state in which the user U wears the heart rate measuring apparatus 1 is expressed as a back side, and a surface on the side opposite from the back side and facing outward is expressed as a front side.

As illustrated in FIG. 1 to FIG. 5, the heart rate measuring apparatus 1 is configured to detect electrocardiac signals generating in association with heart beats by being attached to a breast region of the user U as a surface of a living body, and allows the detected electrocardiac signals to be transmitted via, for example, radio communication. The heart rate measuring apparatus 1 includes the heart rate meter body 2 and a back side belt 4 attached to both ends of the heart rate meter body 2 via coupling members 3.

The back side belt 4 is configured to be capable of surrounding the entire circumference of the breast region of the user U in cooperation with the heart rate meter body 2.

### Heart Rate Meter Body

The heart rate meter body 2 includes a case 5 elongated in the circumferential direction of the breast region of the user U, and body belts 7a, 7b are coupled to both ends of the case 5 via a packing 6. Electrodes 13a and 13b are integrally provided respectively on the back sides of the body belts 7a, 7b (the right side in FIG. 5). A pair of the electrodes 13a and 13b come into contact with the breast region of the user U as the surface of a living body, detect a potential difference occurring between a pair of the electrodes 13a and 13b, and output the detected potential difference as the electrocardiac signal.

The respective electrodes 13a and 13b are formed with circuit connecting portions 14a and 14b respectively at ends thereof on the side of the so as to extend therefrom.

In contrast, the case 5 is formed with depressions 15a and 15b at the both ends thereof so as to accommodate the circuit connecting portions 14a and 14b, and the circuit connecting portions 14a and 14b are mounted in the depressions 15a and 15b. The circuit connecting portions 14a and 14b are formed respectively with insertion holes 17a and 17b which allow insertion of electrode connecting members 16 such as screws or the like at centers thereof and the depressions 15a and 15b are formed with female screw portions 23a and 23b on bottom surfaces thereof at positions corresponding to the insertion holes 17a and 17b. A pair of the electrodes 13a and 13b are fixedly secured to the case 5 together with a circuit board 12, described rater, by the electrode connecting members 16 inserted through the insertion holes 17a and 17b respectively and screwed into the female screw portions 23a and 23b.

The case 5 is formed with a cell housing 8 on the back side thereof at a center in the longitudinal direction thereof, and a button-type cell 9 is housed therein. Furthermore, a cell cover 10 is fixedly secured to the cell housing 8 of the case 5 with screws 11 so as to close the cell housing 8 in which the cell 9 is stored from above.

In contrast, a cover glass 21 configured to cover the front side of the case 5 so as to to define a space K with the case 5 is provided on the front side of the case 5 (the left side in FIG. 5). The cover glass 21 is mounted on the case 5 via the packing 6 and is configured to minimize the probability of entry of moisture such as sweat into the space K. The cover glass 21 is formed so that an outer front surface thereof is flush with the body belts 7a, 7b, and improves its appearance.

The circuit board 12 is arranged in the space K, and is mounted on the front side of the case 5. A terminal 22a provided on the circuit board 12 comes into contact with a negative pole of the cell 9, and a terminal 22b comes into contact with a positive pole of the cell 9.

### Circuit Board

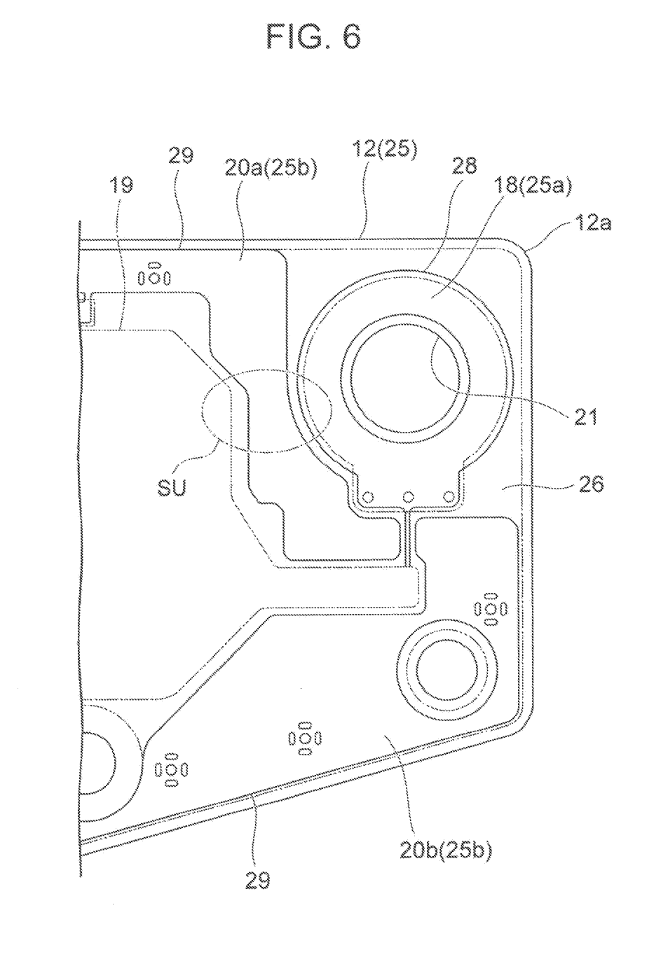

FIG. 6 is a partial plan view of the circuit board 12.

As illustrated in FIG. 5 and FIG. 6, the circuit board 12 is formed into a substantially rectangular shape in plan view so as to correspond to the outline shape of the case 5. Electrode connecting patterns 18 are formed at both ends of the circuit board 12 in the longitudinal direction at positions corresponding to the circuit connecting portions 14a and 14b of the electrodes 13a and 13b. A detection circuit unit 19 configured to measure the heart rate on the basis of the electrocardiac signal detected by the electrodes 13a and 13b is provided over the most part of the center of the circuit board 12. Furthermore, the circuit board 12 is provided with GND patterns (VSS pattern) 20a and 20b between the electrode connecting patterns 18 and the detection circuit unit 19.

The electrode connecting patterns 18 are formed at corner portions 12a of the circuit board 12 and are formed into a substantially circular ring shape in plan view. Through holes 24 are formed at center portions of the electrode connecting patterns 18, and the electrode connecting members 16 are inserted through the through holes 21. In other words, the circuit board 12 is secured together with the circuit connecting portions 14a and 14b of a pair of the electrodes 13a and 13b to the case 5 with the electrode connecting members 16. The electrode connecting patterns 18 of the circuit board 12 and the circuit connecting portions 14a and 14b of a pair of the electrodes 13a and 13b are electrically connected via the electrode connecting members 16.

The GND patterns 20a and 20b are formed over the entire area except for areas where the electrode connecting patterns 18 and the detection circuit unit 19 are formed, and are consequently arranged so that parts of the GND patterns 20a and 20b are respectively arranged between the electrode connecting patterns 18 and the detection circuit unit 19. In other words, the GND patterns 20a and 20b are formed in the periphery of the electrode connecting patterns 18. The GND patterns 20a and 20b are connected to the terminal 22a which comes into contact with the negative pole of the cell 9.

Various electronic components are mounted on the detection circuit unit 19, so that in addition to measurement of heart rate on the basis of the electrocardiac signal detected by a pair of the electrodes 13a and 13b, display of the heart rate, and radio transmission of the heart rate to, for example, a watch or the like, with information of the heart rate as a signal are enabled.

Here, predetermined patterns (not illustrated) of the electrode connecting patterns 18, the GND patterns 20a and 20b, and the detection circuit unit 19 formed on the circuit board 12 are formed as follows.

In other words, a substrate formed with pattern layers 25a and 25b on a front surface of a substrate body 25 and formed with a resist 20 on the pattern layers 25a and 25b is used as the circuit board 12. Portions of the substrate body 25 corresponding to the electrode connecting patterns 18 are set as electrode connecting pattern forming ranges 28, and portions corresponding to the GND patterns 20a and 20b are set as GND pattern forming ranges 29.

By removing the resist 26 in the electrode connecting pattern forming ranges 28 and the resist 26 in the GND pattern forming ranges 28, the pattern layers 25a and 25b are exposed. The exposed pattern layer 25a is used as the electrode connecting patterns 18, and the exposed pattern layer 25b is used as the GND patterns 20a and 20b.

From the two electrode connecting patterns 18 formed in this manner, one of the electrode connecting patterns 18 to which the electrode 13a is connected is connected to the GND patterns 20a and 20b. The electrode connecting pattern 18 to which the other electrode 13b is connected is connected to a predetermined pattern of the detection circuit unit 19.

Here, parts of the GND patterns 20a and 20b are respectively arranged between the electrode connecting patterns 18 and the detection circuit unit 19, and the electrode connecting pattern 18 here means the electrode connecting pattern 18 to which the other electrode 13b is connected, that is, the electrode connecting pattern 18 connected to the predetermined pattern of the detection circuit unit 19.

In this manner, the one electrode 13a is connected to the GND patterns 20a and 20b, and the other electrode 13b is connected to the predetermined pattern of the detection circuit unit 19, so that a potential difference is generated between a pair of the electrodes 13a and 13b. By using the potential difference, the electrocardiac signal is detected. In addition to measurement of heart rate on the basis of the electrocardiac signal, display of the heart rate, and radio transmission of the heart rate to, for example, a watch or the like, with information of the heart rate as a signal are enabled. The detection circuit unit 19 will be described in detail below.

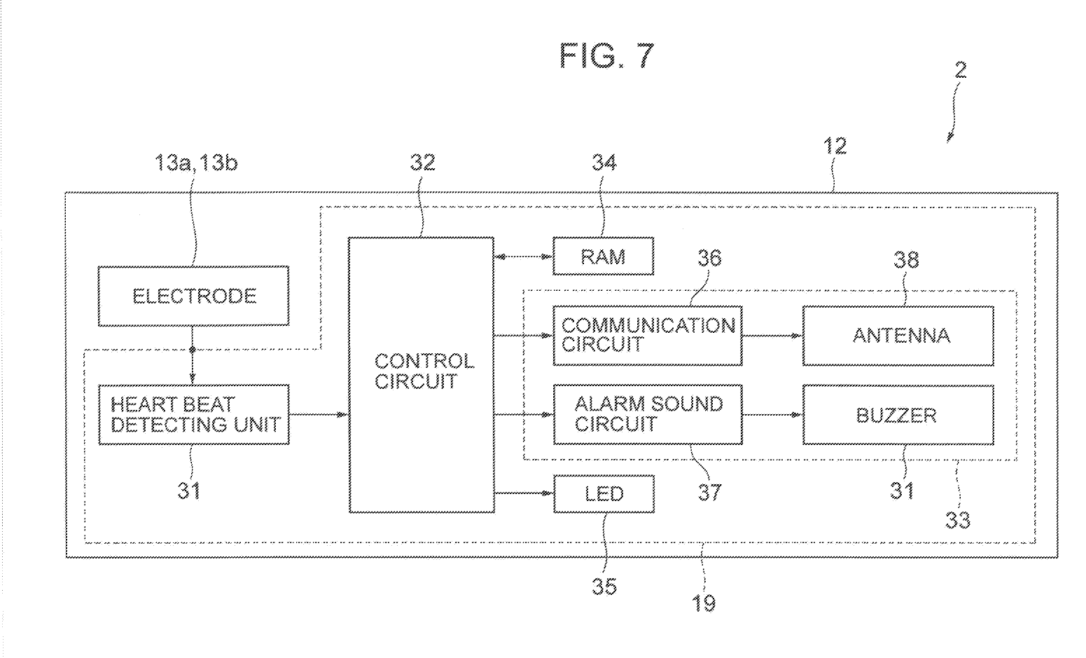

FIG. 7 is a block diagram of the heart rate meter body 2.

As illustrated in FIG. 7, the detection circuit unit 19 includes a heart beat detecting unit 31 configured to detect the heart beat signal on the basis of the electrocardiac signal detected by a pair of the electrodes 13a and 13b, a control circuit unit 32 responsible for controlling, and an alarm unit 33.

The control circuit unit 32 includes a computing unit (not illustrated) configured to process the heart beat signal of the heart beat detecting unit 31 and calculate the heart rate.

A RAM (Random Access Memory) 34 and an LED (Light Emitting Diode) 35 are connected to the control circuit unit 32. The RAM 34 memorizes target values for the comparison with heart rates and various data for each of the heart rates. The LED 35 is used for displaying current heart rate or other heart beat information. Examples of other information include an upper limit value of the heart rate and a lower limit value of the heart rate.

The alarm unit 33 includes a communication circuit 36 configured to transmit the heart beat information via radio communication and an alarm sound circuit 37 configured to perform voice notification. The communication circuit 36 is connected to an antenna 38 mounted en the circuit board 12. The alarm sound circuit 37 is connected so a buzzer 39 mounted on the circuit board 12.

### Operation of GND Pattern

Subsequently, an operation of the GND patterns 20a and 20b will be described with reference to FIG. 5 and FIG. 6.

As illustrated in FIG. 5 and FIG. 6, when, moisture Su (see double-dashed chain line in FIG. 6) enters the space K of the heart rate meter body 2 by being exposed to water or the like, the moisture Su may adhere thereto so as to extend across the detection circuit unit 19 on the circuit board 12 and the electrode connecting pattern 18 connected to the detection circuit unit 19.

Here, if the GND patterns 20a and 20b are not provided on the circuit board 12, there is a case where an electric current flows between a pair of the electrodes 13a and 13b due to the potential difference between a pair of the electrodes 13a and 13b.

In contrast, when the GND patterns 20a and 20b are provided between the detection circuit unit 19 and the electrode connecting pattern 18, even when the moisture Su is adhered thereto so as to extend across the detection circuit unit 19 and the electrode connecting pattern 18, the moisture Su adheres either one of the two GND patterns 20a and 20b (to the GND pattern 20a in FIG. 6) without fail. Therefore, the electrode connecting pattern 18 connected to the detection circuit unit 19 has the same potential as the GND patterns 20a and 20b.

Since the electrode connecting pattern 18 connected to the detection circuit unit 19 has the same potential as the GND patterns 20a and 20b, the other electrode 13b has the same potential with the GND patterns 20a and 20b. Here, since the one electrode 13a is connected to the GND patterns 20a and 20b, the potentials of the respective electrodes 13a and 13b become equivalent.

### Advantages

Therefore, according to the embodiment described above, since the GND patterns 20a and 20b are formed between the detection circuit unit 19 and the electrode connecting pattern 18, for example, even when the moisture Su adheres so as to extend across the detection circuit unit 19 and the electrode connecting pattern 18, the potentials of the respective electrodes 13a and 13b are equivalent, and hence an electric current is prevented from flowing between a pair of the electrodes 13a and 13b. Therefore, the occurrence of malfunction in the heart rate measuring apparatus 1 is prevented.

Since the GND patterns 20a and 20b are formed entirely over the circuit board 12 except for the portion on which the electrode connecting pattern 18 and the detection circuit unit 19 are formed, the moisture Su extending across the detection circuit unit 19 and the electrode connecting pattern 18 can be brought into contact with any one of the GND patterns 20a and 20b without fail. Therefore, the potentials of the respective electrodes 13a and 13b are equalized to prevent an electric current from flowing between a pair of the electrodes 13a and 13b without fail.

Furthermore, by removing the resist 26 in the electrode connecting pattern forming ranges 28 set on the substrate body 25 and the resist 26 in the GND pattern forming ranges 29 when forming the electrode connecting pattern 18 and the GND patterns 20a and 20b, the pattern layers 25a and 25b are exposed and the exposed pattern layer 25a is used as the electrode connecting pattern 18 and the exposed pattern layer 25b is used as the GND patterns 20a and 20b. Therefore, the respective patterns 18, 20a, and 20b may be formed easily.

The invention is not limited to the embodiments shown above, and includes various modifications without departing the scope of the invention.

For example, in the embodiment described above, the case where the GND patterns 20a and 20b are formed over the entire part of the circuit board 12 except for the portions where the electrode connecting pattern 18 and the detection circuit unit 19 are formed has been described. However, the invention is not limited thereto, and the GND patterns 20a and 20b only have to be formed at least between the electrode connecting pattern 18 and the detection circuit unit 19.

In the embodiment described above, the case where the electrode connecting patterns 18 are arranged at the corner portions 12a of the circuit board 12, and the GND patterns 20a and 20b are formed between the electrode connecting patterns 18 and the detection circuit unit 19 has been described. However, the invention is not limited thereto and, for example, the GND pattern only has to be formed around the electrode connecting pattern 18 in the case where the electrode connecting patterns 18 are formed on the inner side of the corner portion 12a of the circuit board 12.

In the embodiment described above, the case where the GND patterns 20a and 20b are connected to the negative pole of the cell 9 via the terminal 22a has been described. However, the invention is not limited thereto, and the low potential pattern may be provided instead of the GND patterns 20a and 20b. The low potential pastern only has to be set, for example, to have a lower potential than the electrode connecting pattern 18 connected to the detection circuit unit 19 by using a resistance or the like.

In the embodiment described above, the case where the heart rate measuring apparatus 1 is configured to be capable of transmitting the detected electrocardiac signal via radio communication has been described. However, the invention is not limited thereto, and may be configured to be capable of performing cable communication.

In the embodiment described above, the case where the circuit board 12 used for the heart rate measuring apparatus 1 configured to measure the heart rate of the user U as the biometric information detecting apparatus is provided with the GND patterns 20a and 20b has been described. However, the invention is not limited thereto, and the GND pattern may be applied to circuit boards used for various biometric information detecting apparatuses. For example, the GND pattern may be applied to a circuit board of an apparatus configured to measure blood pressures, body temperatures, muscle potential and the like as the biometric information detecting apparatus.

What is claimed is:

1. A biometric information detecting apparatus comprising:
a pair of electrodes coming into contact with a surface of a living body; and
a circuit board to which a pair of the electrodes are connected and configured to detect biometric information on the basis of a potential difference generated between a pair of the electrodes, the circuit board including an electrode connecting pattern to which a pair of the electrodes are electrically connected, and a detection circuit unit configured to detect the biometric information on the basis of the potential difference; and
a low potential pattern set to have a potential lower than that of the electrode connecting pattern provided on the circuit board between the electrode connecting pattern and the detection circuit unit.

2. The biometric information detecting apparatus according to claim 1, wherein the low potential pattern is set to a GND pattern.

3. The biometric information detecting apparatus according to claim 1, wherein the low potential pattern is provided in the periphery of the electrode connecting pattern.

4. The biometric information detecting apparatus according to claim 2, wherein the low potential pattern is provided in the periphery of the electrode connecting pattern.

5. The biometric information detecting apparatus according to claim 1, wherein the circuit board is formed by providing a resist on a pattern layer formed on a substrate body, and
a portion on the circuit board corresponding to the low potential pattern is set to be a low potential pattern forming range, and the pattern layer exposed on the circuit board is defined as the low potential pattern by removing the resist in the low potential pattern forming range or by forming the resist in the low potential pattern forming range to be thinner than other portions.

6. The biometric information detecting apparatus according to claim 2, wherein the circuit board is formed by providing a resist on a pattern layer formed on a substrate body, and
a portion on the circuit board corresponding to the low potential pattern is set to be a low potential pattern forming range, and the pattern layer exposed on the circuit board is defined as the low potential pattern by removing the resist in the low potential pattern forming range or by forming the resist in the low potential pattern forming range to be thinner than other portions.

7. The biometric information detecting apparatus according to claim 3, wherein the circuit board is formed by providing a resist on a pattern layer formed on a substrate body, and
a portion on the circuit board corresponding to the low potential pattern is set to be a low potential pattern forming range, and the pattern layer exposed on the circuit board is defined as the low potential pattern by removing the resist in the low potential pattern forming range or by forming the resist in the low potential pattern forming range to be thinner than other portions.

8. The biometric information detecting apparatus according to claim 4, wherein the circuit board is formed by providing a resist on a pattern layer formed on a substrate body, and
a portion on the circuit board corresponding to the low potential pattern is set to foe a low potential pattern forming range, and the pattern layer exposed on the circuit board is defined as the low potential pattern by removing the resist in the low potential pattern forming range or by forming the resist in the low potential pattern forming range to be thinner than other portions.
